(19) United States
(12) Patent Application Publication
(10) Pub. No.: US 2005/0059941 A1
(43) Pub. Date: Mar. 17, 2005

(54) Absorbent product with improved liner treatment

(75) Inventors: Stephen Baldwin, Menasha, WI (US); Duane G. Krzysik, Appleton, WI (US); Bozena Nogaj, Appleton, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/660,319

(22) Filed: Sep. 11, 2003

(51) Int. Cl. A61F 13/15; A61F 13/20

(52) U.S. Cl. 604/367

(57) ABSTRACT

Absorbent products, such as disposable diapers and incontinence garments, comprising an improved lotion formulation are disclosed. The lotion formulation is applied to the bodyfacing surface of the bodyside liner and is stable at elevated temperatures, remains on or near the surface of the absorbent product prior to use, and readily transfers to the user's skin upon use. The lotion formulations described herein have a melt point viscosity of from about 5000 cPs to about 1,000,000 cPs, and a process temperature viscosity of from about 50 cPs to about 50,000 cPs. The lotion formulations comprise an emollient, a structurant, a rheology enhancer, and other optional components.

### BACKGROUND OF THE INVENTION

The present invention relates to absorbent products for absorbing body fluids and exudates such as urine and feces. More particularly, the present invention relates to diapers, training pants, incontinence garments and the like comprising a lotion formulation on the surface thereof comprising an emollient, a structurant, and a rheology enhancer. The novel lotion formulation has improved stability and enhanced aesthetics, and transfers easily from the absorbent product to skin upon use.

Conventional absorbent articles, such as disposable diapers and incontinence garments, typically utilize absorbent materials located between a liquid pervious bodyside liner and a liquid impermeable outer cover to absorb body exudates. Such conventional absorbent articles have typically included elasticized waistbands and leg cuffs to help reduce the leakage of body exudates. Some conventional absorbent articles have also included elasticized containment flaps or barrier flaps at the leg or waist sections of the article to further reduce leaks.

Typically, the liquid pervious bodyside liners have been constructed of nonwoven materials such as spunbond polyolefin materials. Unfortunately, such materials do not always provide a soft, non-abrasive contact surface with the skin. In particular, during continuous use of absorbent articles comprised of such liners, the wearer's skin can become irritated and red, particularly in the presence of urine and/or feces. The abrasion resulting from such liners and the presence of urine and feces can undesirably lead to the onset of diaper dermatitis, commonly known as diaper rash. Diaper dermatitis can afflict almost every infant at some point in time during the diaper wearing years. Although others factors influence the onset of diaper dermatitis, critical factors include the abrasiveness of the bodyside liner and the hydration level of the wearer's skin.

To prevent body exudates from contracting the wearer's skin, the caregiver often applies skin protective products directly to the skin of the wearer before positioning the article on the wearer. Such products have included petrolatum, mineral oil, talc, corn starch, or various other commercially available rash creams or lotions. This procedure typically involves the caregiver applying the products to their hand and then transferring the product to the wearer's skin.

To eliminate the caregiver from contacting the products and to reduce skin abrasion and improve skin health, lotion formulations can be applied to the bodyside liner such that, during use, the lotion formulation either transfers to the skin or provides lubricity thereby reducing the friction between the liner and the skin. Conventional lotion formulations have typically been lipophilic liquids, lipophilic semisolids, or lipophilic solids based formulations at room temperature. Such formulations have been unstable and tended to migrate away from the surface of the liner into the liner and absorbent core of the absorbant articles leaving less lotion on the surface to transfer to the skin or provide the reduced abrasion. This migration problem is particularly evident at higher temperatures such as those encountered in typical storage or transportation.

In an attempt to counteract the potential for the lotion to migrate into the product and away from the outer surface where it can provide the intended benefit, some have simply increased the amount of lotion applied to the absorbent articles to ensure a satisfactory amount remains on the surface. Although this may increase the amount of lotion on the surface of the absorbent article, it can also make the article greasy or wet to the touch, which is highly undesirable. Such an increased addition to the product may also require special packaging to ensure that there is no leakage. This can increase costs.

Because of the potential migration and transfer problems described above, conventional absorbent articles have not been completely satisfactory. As such, a need exists in the industry for a lotioned absorbent article with improved stability.

### SUMMARY OF THE INVENTION

The present invention provides absorbent products such as diapers and incontinence garments comprising an improved lotion formulation on the surface thereof. The lotion formulation, which may conveniently be applied to the bodyfacing surface of the bodyside liner of the absorbent product, is stable at elevated temperatures, remains on or near the surface of the absorbent product prior to use, and readily transfers to the user's skin upon use. The lotion formulations described herein have a melt point viscosity as defined herein of from about 5000 cPs to about 1,000,000 cPs, and a process temperature viscosity as defined herein of from about 50 cPs to about 50,000 cPs.

Specifically, the lotion formulations comprise the following components:
(a) an emollient;
(b) a structurant;
(c) a rheology enhancer; and
(d) other optional components.

Other optional components suitable for use in the lotion formulations described herein include, for example, moisturizers, vitamins, botanical extracts, skin protectants, astringents, lipids, sterols, powders, fragrances, antioxidants, colorants, preservatives, fragrances, optical brighteners, sunscreens, alpha hydroxy acids, and combinations thereof. Additionally, a hydrophilic surfactant may be utilized to emulsify various ingredients into the formulation, and improve wettability of the product.

In a preferred embodiment, the rheology enhancer component of the lotion formulation is selected from the group consisting of dextrin palmitate, dextrin palmitate ethylhexanoate, stearoyl inulin, ethylene/propylene/styrene copolymers alone or in combination with mineral oil or petrolatum, butylene/ethylene/styrene copolymers alone or in combination with mineral oil or petrolatum, styrene/butadiene/styrene copolymers, styrene/isoprene/styrene copolymers, styrene-ethylene/butylene-styrene copolymers, styrene-ethylene/propylene-styrene copolymers, (styrene-butadiene)n polymers, (styrene-isoprene)n polymers, styrene-butadiene polymers, styrene-ethylene/propylene copolymers, polyethylene polyisobutylenes, polyisobutylenes, polyisobutenes, and combinations thereof.

Briefly, therefore, the present invention is directed to an absorbent article comprising a liner material having a bodyfacing surface. The bodyfacing surface has deposited thereon a lotion formulation in an amount of from about 0.05 mg/cm² to about 100 mg/cm². The lotion formulation comprises from about 10% (by total weight of the formulation) to about 89% (by total weight of the formulation) of an emollient, from about 10% (by total weight of the formulation) to about 50% (by total weight of the formulation) of a structurant, and from about 0.1% (by total weight of the formulation) to about 40% (by total weight of the formulation) of a rheology enhancer. The rheology enhancer is selected from the group consisting of dextrin palmitate, dextrin palmitate ethylhexanoate, stearoyl inulin, ethylene/propylene/styrene copolymers alone or in combination with mineral oil or petrolatum, butylene/ethylene/styrene copolymers alone or in combination with mineral oil or petrolatum, styrene/butadiene/styrene copolymers, styrene/isoprene/styrene copolymers, styrene-ethylene/butylene-styrene copolymers, styrene-ethylene/propylene-styrene copolymers, (styrene-butadiene) n polymers, (styrene-isoprene) n polymers, styrene-butadiene polymers, styrene-ethylene/propylene copolymers, polyethylene polyisobutylenes, polyisobutylenes, polyisobutenes, and combinations thereof.

The present invention is further directed to an absorbent article comprising a liner material having a bodyfacing surface. The bodyfacing surface has deposited thereon a lotion formulation in an amount of from about 0.05 mg/cm² to about 100 mg/cm². The lotion formulation comprises from about 10% (by total weight of the formulation) to about 89% (by total weight of the formulation) of an emollient, from about 10% (by total weight of the formulation) to about 50% (by total weight of the formulation) of a structurant, and from about 0.1% (by total weight of the formulation) to about 40% (by total weight of the formulation) of a rheology enhancer. The lotion formulation has a melt point viscosity of from about 5000 cPs to about 1,000,000 cPs and a process temperature viscosity of from about 50 cPs to about 50,000 cPs. The rheology enhancer is selected from the group consisting of dextrin palmitate, dextrin palmitate ethylhexanoate, stearoyl inulin, ethylene/propylene/styrene copolymers alone or in combination with mineral oil or petrolatum, butylene/ethylene/styrene copolymers alone or in combination with mineral oil or petrolatum, styrene/butadiene/styrene copolymers, styrene/isoprene/styrene copolymers, styrene-ethylene/butylene-styrene copolymers, styrene-ethylene/propylene-styrene copolymers, (styrene-butadiene)n polymers, (styrene-isoprene)n polymers, styrene-butadiene polymers, styrene-ethylene/propylene copolymers, polyethylene polyisobutylenes, polyisobutylenes, polyisobutenes, and combinations thereof.

Other features and advantages of this invention will be in part apparent and in part pointed out hereinafter.

### BRIEF DESCRIPTION OF THE DRAWINGS

FIG. 1 representatively shows a partially cut away top plan view of an absorbent article in a stretched and laid flat condition with the surface of the article which contacts the skin of the wearer facing the viewer.

FIG. 2 representatively shows a sectional view of the absorbent article of FIG. 1 taken along line 2-2.

FIG. 3 representatively shows a top plan view of the bodyside liner of the absorbent article of FIG. 1 with the surface, which contacts the wearer facing the viewer.

### DETAILED DESCRIPTION OF THE PREFERRED EMBODIMENTS

In accordance with the present invention, it has been discovered that specific rheology enhancers can be introduced into lotion formulations for use in combination with an absorbent product, such as a diaper, incontinence garment, and the like, to provide a lotion formulation that remains on the surface of the absorbent product and does not substantially migrate into the interior of the product prior to use. Surprisingly, the rheology enhancers, which impart a specific melt point viscosity and process temperature viscosity to the lotion formulation to significantly improve performance, do not substantially negatively affect the transfer of the lotion formulation to the skin during use.

The lotion formulations of the present invention are described herein in combination with a disposable diaper. It is readily apparent, however, that the lotion formulations of the present invention are equally applicable to other disposable absorbent products such as feminine care pads, incontinence garments, training pants, swimming pants, and the like.

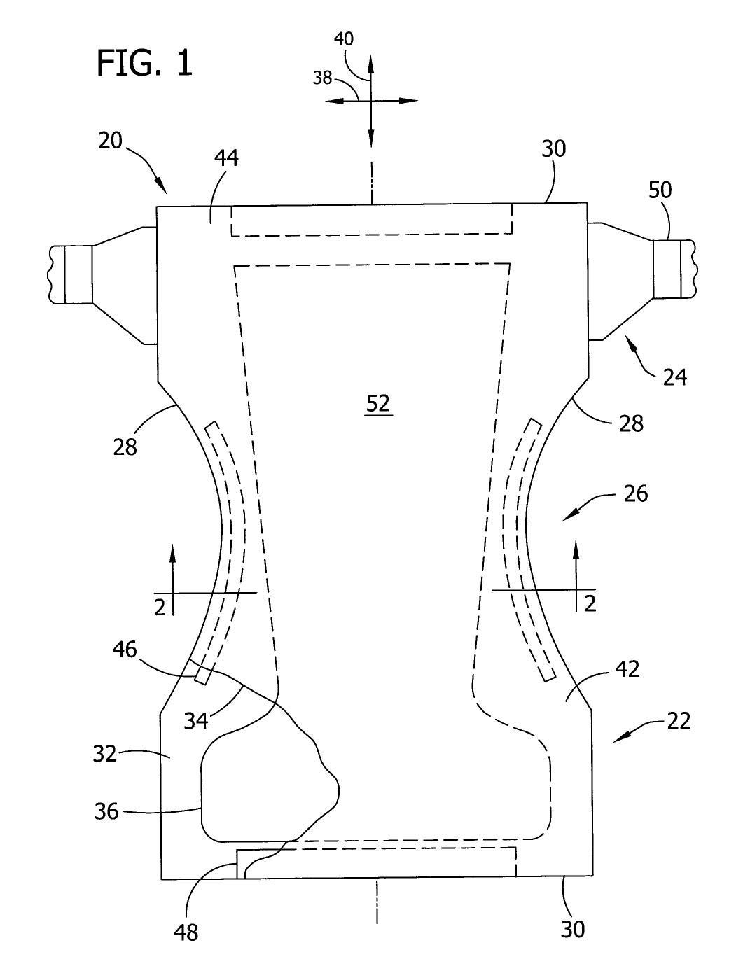

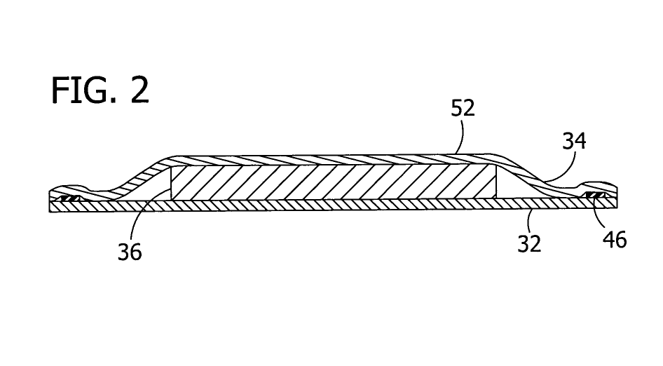

With reference to FIGS. 1 and 2, an integral absorbent garment article, such as a disposable diaper 20, generally defines a front waist section 22, a rear waist section 24, an intermediate section 26 which interconnects the front and rear waist section, a pair of laterally opposed side edges 28, and a pair of longitudinally opposed end edges 30. The front and rear waist sections include the general portions of the article which are constructed to extend substantially over the wearer's front and rear abdominal regions, respectively, during use. The intermediate section of the article includes the general portion of the article which is constructed to extend through the wearer's crotch region between the legs. The opposed side edges 28 define leg openings for the diaper and generally are curvilinear or contoured to more closely fit the legs of the wearer. The opposed end edges 30 define a waist opening for the diaper 20 and typically are straight but may also be curvilinear.

FIG. 1 is a representative plan view of the diaper 20 in a flat, non-contracted state. Portions of the structure are partially cut away to more clearly show the interior construction of the diaper 20, and the surface of the diaper which contacts the wearer is facing the viewer. The diaper 20 includes a substantially liquid impermeable outer cover 32, a porous, liquid permeable bodyside liner 34 positioned in facing relation with the outer cover 32, and an absorbent body 36, such as an absorbent pad, which is located between the outer cover and the bodyside liner. The diaper 20 also defines a lateral direction 38 and a longitudinal direction 40. Marginal portions of the diaper 20, such as marginal sections of the outer cover 32, may extend past the terminal edges of the absorbent body 36. In the illustrated embodiment, for example, the outer cover 32 extends outwardly beyond the terminal marginal edges of the absorbent body 36 to form side margins 42 and end margins 44 of the diaper 20. The bodyside liner 34 is generally coextensive with the outer cover 32, but may optionally cover an area which is larger or smaller than the area of the outer cover 32, as desired.

To provide improved fit and to help reduce leakage of body exudates from the diaper 20, the side margins 42 and end margins 44 of the diaper may be elasticized with suitable elastic members, such as leg elastic members 46 and waist elastic members 48. For example, the leg elastic members 46 may include single or multiple strands of elastic or elastomeric composites which are constructed to operably gather and shirr the side margins 42 of the diaper 20 to provide elasticized leg bands which can closely fit around the legs of the wearer to reduce leakage and provide improved comfort and appearance. Similarly, the waist elastic members 48 can be employed to elasticize the end margins 44 of the diaper 20 to provide elasticized waistbands. The waist elastics are configured to operably gather and shirr the waistband sections to provide a resilient, comfortably close fit around the waist of the wearer.

The elastic members 46 and 48 are secured to the diaper 20 in an elastically contractible condition so that in a normal under strain configuration, the elastic members effectively contract against the diaper 20. For example, the elastic members 46 and 48 may be elongated and secured to the diaper 20 while the diaper is in an uncontracted condition. In FIGS. 1 and 2, the elastic members 46 and 48 are illustrated in their uncontracted, stretched condition for the purpose of clarity. The diaper 20 may also include a pair of elasticized, longitudinally extending containment flaps (not shown), which are configured to maintain an upright, perpendicular arrangement in at least the intermediate section 26 of the diaper 20 to serve as an additional barrier to the lateral flow of body exudates. Suitable constructions and arrangements of containment flaps are well known to those skilled in the art.

Alternatively, the diaper 20 may include a pair of separate, elasticized and gathered leg gussets (not shown) or combination leg gussets/containment flaps (not shown) which are attached to the diaper along the side margins 42 in at least the intermediate section 26 of the diaper 20 to provide elasticized leg cuffs. Such gussets or combination gussets/containment flaps may be configured to extend beyond and bridge across the respective concave portion of the side margins 42.

The diaper 20, as representatively illustrated in FIGS. 1 and 2, may further include a pair of fasteners 50 employed to secure the diaper 20 about the waist of a wearer. Suitable fasteners 50 include hook-and-loop type fasteners, adhesive tape fasteners, buttons, pins, snaps, mushroom-and-loop fasteners, and the like. A cooperating side panel member can be associated with each fastener and may be constructed to be nonelasticized, or to be elastically stretchable at least along the lateral direction 38 of diaper 20.

The diaper may further include a surge management layer (not shown) positioned between the bodyside liner 34 and the absorbent body 36 which is configured to efficiently hold and distribute liquid exudates to the absorbent body 36. The surge management layer can prevent the liquid exudates from pooling and collecting on the portion of the diaper positioned against the wearer's skin, thereby reducing the level of skin hydration. Suitable constructions and arrangements of surge management layers are well known to those skilled in the art. Other suitable diaper components may also be incorporated on absorbent articles described herein.

The diaper 20 may be of various suitable shapes. For example, the diaper may have an overall rectangular shape, T-shape, or an approximately hour-glass shape. In the shown embodiment, the diaper 20 is I-shaped. Examples of diaper configurations suitable for use in connection with the instant application and other diaper components suitable for use on diaper 20 are described in U.S. Pat. Nos. 4,798,603 issued Jan. 17, 1989 to Meyer et al.; U.S. Pat No. 5,176,668 issued Jan. 5, 1993, to Bernardin; U.S. Pat No. 5,176,672 issued Jan. 5, 1993 to Bruemmer et al.; U.S. Pat. No. 5,192,606 issued Mar. 9, 1993 to Proxmire et al.; and U.S. Pat. No 5,509,915 issued Apr. 23, 1996 to Hanson et al., the disclosures of which are hereby incorporated by reference. The various aspects and configuration of the invention can provide distinctive combinations of softness, body conformity, reduced red-marking of the wearer's skin, reduced hydration, and improved containment of body exudates.

The various components of the diaper 20 are integrally assembled together employing various types of suitable attachment means, such as adhesive, sonic bonds, thermal bonds, or combinations thereof. In the shown embodiment, for example, the bodyside liner 34 and the outer cover 32 are assembled to each other and to the absorbent body 36 with adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Similarly, other diaper components, such as the elastic members 46 and 48 and the fasteners 50, may be assembled into the diaper 20 by employing the above-identified attachment mechanisms.

The outer cover 32 of the diaper 20, as representatively illustrated in FIGS. 1 and 2, may suitably be composed of material which is either liquid permeable or liquid impermeable. It is generally preferred that the outer cover 32 be formed from a material which is substantially impermeable to liquids. For example, a typical outer cover can be manufactured from a thin plastic film or other flexible liquid-impermeable material. For example, the outer cover 32 may be formed from a polyethylene film having a thickness of from about 0.012 millimeter (0.5 mil) to about 0.051 millimeter (2.0 mils). If it is desired to present the outer cover with a more clothlike feeling, the outer cover 32 may comprise a polyolefin film having a nonwoven web laminated to the outer surface thereof, such as a spunbond web of polyolefin fibers. For example, a stretch-thinned polypropylene film having a thickness of about 0.015 millimeter (0.6 mils) may have thermally laminated thereto a spunbond web of polypropylene fibers, which fibers have a thickness of about 1.5 to 2.5 denier per filament, which nonwoven web has a basis weight of about 17 grams per square meter (0.5 ounce per square yard). Methods of forming such clothlike outer covers are known to those skilled in the art. Further, the outer cover 32 may be formed of a woven or nonwoven fibrous web layer which has been totally or partially constructed or treated to impart a desired level of liquid impermeability to selected regions that are adjacent or proximate to the absorbent body 36.

Desirably, the outer cover 32 may be composed of a “breathable” material which permits vapors to escape from the absorbent body 36 while still preventing liquid exudates from passing through the outer cover 32. For example, the outer cover 20 is desirably constructed to be permeable to at least water vapor and has a water vapor transmission rate of at least about 1000 g/m²/24 hours, desirably at least about 1500 g/m²/24 hours, more desirably at least about 2000g/m²/24 hours, and even more desirably at least about 3000 g/m²/24 hours. Materials which have a water vapor transmission rate less than those above do not allow a sufficient amount of air exchange and undesirably result in increased levels of skin hydration. As used herein, the phrase “water vapor transmission rate” (WVTR) refers to the WVTR value according to the Water Vapor Transmission Rate Test which is described in further detail herein below.

In a particular embodiment, the outer cover 20 is provided by a microporous film/nonwoven laminate material comprising a spunbond nonwoven material laminated to a microporous film. For example, the laminate may include a 0.6 osy (20.4 gsm) polypropylene spunbond material thermally attached to a 18.7 gsm stretched microporous film. The film may include from about 20 percent to about 75 percent by weight calcium carbonate particulates and the remainder primarily low density polyethylene. The film is then stretched which causes the polyethylene component to stretch while the particulates remain unstretched, thus causing voids to develop around the calcium carbonate particles in the film. The resulting laminate may define a water vapor transmission rate of from about 1000 to about 5000 g/m²/24 hours.

Examples of suitable breathable materials for the outer cover 20 are also described in U.S. Pat. No. 5,879,341 issued Mar. 9, 1999 to Odorzynski et al. and entitled “ABSORBENT ARTICLE HAVING A BREATHABILITY GRADIENT”; U.S. Pat. No. 5,843,056 issued Dec. 1, 1988, to Good et al. and entitled ABSORBENT ARTICLE HAVING A COMOSITE BREATHABLE OUTER COVER”; and U.S. Pat. No. 5,855,999 issued Jan. 5, 1999 to McCormack et al. and entitled “BREATHABLE, CLOTH-LIKE FILM/NONWOVEN COMPOSITE”, the disclosures of which are herein incorporated by reference.

The absorbent body 36 of the diaper 20, as representatively illustrated in FIGS. 1 and 2, may suitable comprise a matrix of hydrophilic fibers, such as a web of cellulosic fluff, mixed with particles of a high-absorbency material commonly known as superabsorbent material. In a particular embodiment, the absorbent body 36 comprises a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be non-uniformly mixed. The fluff and superabsorbent particles may also be selectively placed into desired zones of the absorbent body 36 to better contain and absorb body exudates. The concentration of the superabsorbent particles may also vary through the thickness of the absorbent body 36. Alternatively, the absorbent body 36 may comprise a laminate of fibrous webs and superabsorbent material or other suitable means of maintaining a superabsorbent material in a localized area.

The absorbent body 36 may have any of a number of shapes. For example, the absorbent core may be rectangular, I-shaped, or T-shaped. It is generally preferred that the absorbent body 36 be narrower in the crotch area than in the front or rear portions of the diaper 20. The size and the absorbent capacity of the absorbent body 36 should be compatible with the size of the intended wearer and the liquid loading imparted by the intended use of the absorbent article.

The high-absorbency material can be selected from natural, synthetic, and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful in the present invention. Such high-absorbency materials are well known to those skilled in the art and are widely commercially available. Examples of superabsorbent polymers suitable for use in the present invention are SANWET IM 3900 polymer available from Hoechst Celanese located in Portsmouth, Va., and DOW DRYTECH 2035LD polymer available from Dow Chemical Company located in Midland, Mich.

The high absorbency material may be in any of a wide variety of geometric forms. As a general rule, it is preferred that the high absorbency material be in the form of discrete particles. However, the high absorbency material may also be in the form of fibers, flakes, rods, spheres, needles, or the like. As a general rule, the high absorbency material is present in the absorbent body in an amount of from about 5 to about 90 weight percent based on a total weight of the absorbent body 36.

Optionally, a substantially hydrophilic tissue wrapsheet (not shown) may be employed to help maintain the integrity of the airlaid fibrous structure of the absorbent body 36. The tissue wrapsheet is typically placed about the absorbent body over at least the two major facing surfaces thereof and composed of an absorbent cellulosic material, such as creped wadding or a high wet-strength tissue. In one aspect of the invention, the tissue wrapsheet can be configured to provide a wicking layer, which helps to rapidly distribute liquid over the mass of absorbent fibers comprising the absorbent body. In another aspect of the invention, the wrapsheet material on one side of the absorbent fibrous mass may be bonded to the wrapsheet located on the opposite side of the fibrous mass.

The bodyside liner 34, as representatively illustrated in FIGS. 1 and 2, suitably presents a bodyfacing surface which is compliant, soft feeling, and non-irritating to the wearer's skin. Further, the bodyside liner 34 may be less hydrophilic than the absorbent body 36, to present a relatively dry surface to the wearer, and may be sufficiently porous to be liquid permeable, permitting liquid to readily penetrate through its thickness. A suitable bodyside liner 34 may be manufactured from a wide selection of web materials, such as porous foams, reticulated foams, apertured plastic films, natural fibers (i.e., wood or cotton fibers), synthetic fibers (i.e., polyester or polypropylene fibers), or a combination of natural and synthetic fibers. The bodyside liner 34 is suitably employed to help isolate the wearer's skin from liquids held in the absorbent body 36.

Various woven and nonwoven fabrics can be used for the bodyside liner 34. For example, the bodyside liner 34 may be composed of a meltblown or spunbonded web of polyolefin fibers. The bodyside liner 34 may also be a bonded-carded web composed of natural and/or synthetic fibers. The bodyside liner 34 may be composed of a substantially hydrophobic material, and the hydrophobic material may, optionally, be treated with a surfactant, a wetting agent, or otherwise processed to impart a desired level of wettability and hydrophilicity.

In a particular embodiment, the bodyside liner 34 comprises a nonwoven, spunbond, polypropylene fabric composed of about 2.8-3.2 denier fibers formed into a web having a basis weight of about 20 grams per square meter and a density of about 0.13 gram per cubic centimeter. The fabric may be surface treated with about 0.3 weight percent of a surfactant mixture, which contains a mixture of AHCOVEL Base N-62 and GLUCOPOAN 220UP surfactant in a 3:1 ratio based on a total weight of the surfactant mixture. The ANCOVEL Base N-62 is purchased from Hodgson Textile Chemicals Inc., (Mount Holly, North Carolina) and includes a blend of hydrogenated ethoxylated castor oil and sorbitan monooleate in a 55:45 weight ratio. The GLUCOPAN 220UP is purchased from Henkel Corporation and includes alkyl polyglycoside. The surfactant may be applied by any conventional means, such as spraying, printing, brush coating, or the like. The surfactant may be applied to the entire bodyside liner 34, or may be selectively applied to particular sections of the bodyside liner 34, such as the medial section along the longitudinal centerline of the diaper, to provide greater wettability of such sections.

The bodyside liner 34 of the absorbent article includes a lotion formulation on the bodyfacing surface 52 thereof. As noted above, the lotion formulations of the present invention for use in combination with an absorbent article comprise an emollient, a structurant, and a rheology enhancer. Other optional components, such as surfactants, may also be included in the lotion formulations as discussed herein.

An emollient is an active ingredient in a formulation that typically softens, soothes, supples, coats, lubricates and/or moisturizes the skin. Generally, emollients accomplish several of these objectives simultaneously. Typically, emollients suitable for use in the lotion formulations described herein are fluids at room temperature such that they impart a soft, lubricious lotion-like feel upon use. The amount of emollient in the lotion formulation is from about 10% (by total weight of the formulation) to about 89% (by total weight of the formulation), desirably from about 30% (by total weight of the formulation) to about 80% (by weight of the formulation), desirably from about 60% (by total weight of the formulation) to about 80% (by total weight of the formulation).

Suitable emollients for use in the lotion formulations of the present invention are typically substantially water-free. Although the emollient may contain trace amounts of water as a contaminant without substantially harming the lotion formulation, it is preferred that the amount of water be less than about 5% by weight of the emollient component of the lotion formulation to reduce the likelihood of microbial growth and product destruction.

Suitable emollients for use in the lotion formulations of the present invention include, for example, petrolatum, mineral oil, mineral jelly, isoparaffins, vegetable oils such as avocado oil, borage oil, canola oil, castor oil, chamomile, coconut oil, corn oil, cottonseed oil, evening primrose oil, safflower oil, sunflower oil, soybean oil, sweet almond, and the like, lanolin, partially hydrogenated vegetable oils, polydimethylsiloxanes such as methicone, cyclomethicone, dimethicone, dimethiconol, and trimethicone, organo-siloxanes (i.e., where the organic functionality can be selected from alkyl, phenyl, amine, polyethylene glycol, amine-glycol, alkylaryl, carboxal, and the like), silicone elastomer, gums, resins, fatty acid esters (esters of C₆-C₂₈ fatty acids and C₆-C₂₈ fatty alcohols), glyceryl esters and derivatives, fatty acid ester ethoxylates, alkyl ethoxylates, C₁₂-C₂₈ fatty alcohols, C₁₂-C₂₈ fatty acids, C₁₂-C₂₈ fatty alcohol ethers, Guerbet alcohols, Guerbet Acids, Guerbet Esters, and other cosmetically acceptable emollients.

Additionally, some emollients are solids at room temperate, and may have a dual benefit of being solid emollients as well as structuring agents. These include, for example, C₁₄-C₂₈ fatty acid esters (esters of C₁-C₂₈ fatty acids, and C₁₂-C₂₈ fatty alcohols), C₁₄-C₂₈ fatty alcohols, C₁₄-C₂₈ fatty acids, C₁₄-C₂₈ fatty acid ethoxylates, C₁₄-C₂₈ fatty ethers and C₁₆-C₃₀ alkyl siloxanes.

To provide the intended benefits in the lotion formulations of the present invention, the emollient component of the lotion formulation is present in an amount of from about 10% (by total weight of the lotion formulation) to about 89% (by total weight of the lotion formulation), desirably from about 30% (by total weight of the lotion formulation) to about 80% (by total weight of the lotion formulation), and even more desirably from about 60% (by total weight of the lotion formulation) to about 80% (by total weight of the lotion formulation). Lotion formulations which include an amount of emollient greater that the recited amounts tend to have lower viscosities which can lead to unwanted migration of the lotion formulation. Lotion formulations which include an amount of emollient less than the recited amounts tend to have poor transfer to the wearer's skin.

The structurant utilized in the lotion formulations described herein help to immobilize the emollient, and other components of the lotion formulation, on the surface of the absorbent product where they are of greatest value. Because some emollients are fluids at room temperature, they may tend to flow or migrate away from the surface of the absorbent product into the interior of the product where they are of limited value and may tend to decrease the absorbency of the absorbant core material of the product due to making the absorbant core hydrophobic. The structurant reduces the ability of the emollient (and other components) from migrating and keeps the emollient primarily on the surface of the absorbent product to improve the transfer of the lotion formulation to the skin of the wearer. In addition to acting as a structurant, some of the specified structurants may also act as emollients, occlusive agents, moisturizers, barrier enhancers, and combinations thereof.

Suitable structurants for use in the lotion formulations disclosed herein have a melting point of about 45° C. to about 85° C. and may include, for example, waxes including animal waxes, vegetable waxes, mineral waxes, synthetic waxes and polymers. Exemplary structurants include bayberry wax, beeswax C30 alkyl dimethicone, candelilla wax, carnauba, ceresin, cetyl esters,stearyl benzoate, behenyl benzoate, esparto, hydrogenated cottonseed oil, hydrogenated jojoba oil, hydrogenated jojoba wax, hydrogenated microcrystalline wax, hydrogenated rice bran wax, japan wax, jojoba buffer, jojoba esters, jojoba wax, lanolin wax, microcrystalline wax, mink wax, motan acid wax, motan wax, ouricury wax, ozokerite paraffin, PEG-6 beeswax, PEG-8 beeswax, rezowax, rice bran wax, shellac wax, spent grain wax, spermaceti wax, steryl dimethicone, synthetic beeswax, synthetic candelilla wax, synthetic carnauba wax, synthetic japan wax, synthetic jojoba wax, C₁₄-C₂₈ fatty alcohols, C₁₄-C₂₈ fatty acids, polyethylene,ethylene vinyl acetate copolymers, ethylene-alpha olefin copolymers, ethylene homopolymers such as Petrolite EP copolymers from Baker Hughes Inc., (Sugar Land TX.), C₁₈-C₄₅ olefins, poly alpha olefins such as Vybar Polymers from Baker Hughes Inc. or Okerin Polymers from Honeywell Specialty Chemicals, (Duluth, GA), hydrogenated vegetable oils, polyhydroxy fatty acid esters, polyhydroxy fatty acid amides, ethoxylated fatty alcohols and esters of C₁₂-C₂₈ fatty acids, and C₁₂-C₂₈ fatty alcohols.

To provide the intended benefits in the lotion formulations of the present invention, the structurant component of the lotion formulation is present in an amount of from about 10% (by total weight of the lotion formulation) to about 50% (by total weight of the lotion formulation), and desirably from about 20% (by total weight of the lotion formulation) to about 40% (by total weight of the lotion formulation). Lotion formulations which include an amount of structurant less than the recited amounts tend to have lower viscosities which undesirably lead to migration of the lotion formulation. Lotion formulations which include an amount of structurant greater than the recited amounts tend to provide less transfer to the wearer's skin.

The rheology enhancers utilized in the lotion formulations increase the melt point viscosity of the lotion formulation so that the formulation readily remains on the surface of the absorbent product and does not substantially migrate into the interior of the absorbent product, while substantially not affecting the transfer of the lotion formulation to the skin. Additionally, the rheology enhancers help the lotion formulation to maintain a high viscosity at elevated temperatures, such as those encountered during storage and transportation. Desirably, the rheology enhancer increases the viscosity of the lotion formulation by at least about 50%, more desirably at least about 500%, and even more desirably at least about 1000%.

Suitable rheology enhancers include dextrin palmitate, dextrin palmitate ethylhexanoate, stearoyl inulin, combinations of alpha-olefins and styrene alone or in combination with mineral oil or petrolatum, combinations of di-functional alpha-olefins and styrene alone or in combination with mineral oil or petrolatum, combinations of alpha-olefins and isobutene alone or in combination with mineral oil or petrolatum, ethylene/propylene/styrene copolymers alone or in combination with mineral oil or petrolatum, butylene/ethylene/styrene copolymers alone or in combination with mineral oil or petrolatum, styrene/butadiene/styrene copolymers, styrene/isoprene/styrene copolymers, styrene-ethylene/butylene-styrene copolymers, styrene-ethylene/propylene-styrene copolymers, (styrene-butadiene)n polymers, (styrene-isoprene)n polymers, styrene-butadiene polymers, styrene-ethylene/propylene copolymers, polyethylene polyisobutylenes, polyisobutylenes, polyisobutenes, and combinations thereof. Particularly preferred rheology enhancers include those sold under the tradename Versagel (Penreco, Houston, Texas).

To provide the intended benefits in the lotion formulations of the present invention, the rheology enhancer component of the lotion formulation is present in an amount of from about 0.1% (by total weight of the lotion formulation) to about 40% (by total weight of the lotion formulation), and desirably from about 0.5% (by total weight of the lotion formulation) to about 30% (by total weight of the lotion formulation), and even more desirably from about 1% (by total weight of the lotion formulation) to about 25% (by total weight of the lotion formulation).

The lotion formulations described herein have specific melt point and process temperature viscosities, as defined herein. These viscosities are important for at least two reasons. First, the higher the melt point or process temperature viscosity, the less likely the lotion formulation is to penetrate into the inner surface of the absorbent product. The less lotion formulation that is able to penetrate into the interior of the absorbent product, results in more lotion formulation on the surface of the product that can transfer to the user's skin. Secondly, the higher the viscosity of the formulation at or above the melting point of the formulation, the less likely the formulation will be to migrate at typical or adverse storage or temperature conditions.

The lotion formulations described herein have a melt point viscosity of from about 5000 cPs to about 1,000,000 cPs, desirably from about 50,000 cPs to about 800,000 cPs, and more desirably from about 100,000 cPs to about 500,000 cPs. As used herein, the term “melt point viscosity” means the viscosity of the formulation at the point in time when the formulation visually becomes a liquid. Lotion formulations having melt point viscosities in these ranges significantly improve the ability of the lotion formulation to remain on the surface of the absorbent product and the formulation maintains a high viscosity at elevated temperatures, such as those encountered during storage and shipment.

Additionally, to improve application to the surface of the absorbent product, the lotion formulations described herein have a process temperature viscosity of from about 50 cPs to about 50,000 cPs, desirably from about 75 cPs to about 10,000 cPs, and more desirably from about 100 cPs to about 5,000 cPs. The process temperature is typically from about 5° C. to about 10° C. above the melting point of the lotion formulation.

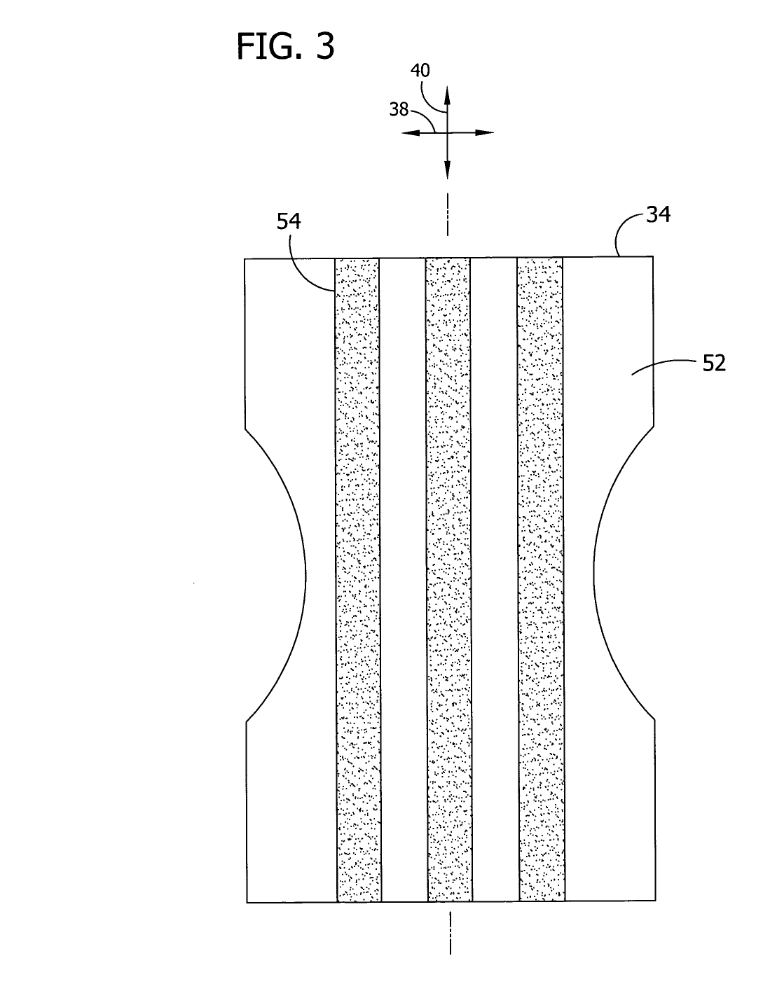

The lotion formulations described herein may be applied to the entire bodyfacing surface 52 of the bodyside liner 34, or may be selectively applied to particular section of the bodyfacing surface 52, such as the medial section along the longitudinal centerline of the diaper, to provide greater lubricity of such sections and to transfer such lotion to the wearer's skin. Alternatively, as representatively illustrated in FIG. 3, the bodyfacing surface 52 of the bodyside liner 34 may include multiple stripes 54 of the lotion formulation applied thereto. For example, the bodyfacing surface 52 of bodyside liner 34 may include from 1 to 10 stripes 54 of lotion formulation extending along the longitudinal direction 40 of the diaper 20. The stripes 54 may extend the full length of the bodyside liner 34 or only a portion thereof. The stripes 54 may also define a width of from about 0.2 to about 1 centimeters.

The lotion formulation should cover a sufficient amount of the surface area of the bodyside liner 34 to ensure adequate transfer to the skin and reduced abrasion between the liner 34 and the wearer's skin. Desirably, the lotion formulation is applied to at least about 5 percent and more desirably at least about 25 percent of the bodyfacing surface 52 of the bodyside liner 34.

The lotion formulation can be applied to the bodyside liner 34 at any add-on level that provides the desired transfer benefit. For example, the total add-on level of the lotion formulation can be from about 0.05 to about 100 mg/cm², desirably from about 1 to about 50 mg/cm², and more desirably from about 10 to about 40 mg/cm² for improved performance. The exact add-on amount will depend upon the desired effect of the lotion on the product attributes and the specific lotion formulation. As mentioned above, the improved stability and reduce tendency to migrate of the lotion formulations of the present invention allows a lesser amount of lotion to be applied to the absorbent product to achieve the same benefit when compared with conventional lotion formulations.

The lotion formulation may be applied to the absorbent product in one of many well known manners. A preferred method to uniformly apply the lotion formulation to the surface of the absorbent product is spraying or slot coating, as these processes are exact and offer maximum control of the formulation distribution and transfer rate. Other known methods, such as rotogravure or flexographic printing, are also suitable.

The lotion formulations described herein have a penetration hardness such that the lotion formulation is stable on the surface of the absorbent product, yet easily transferred to the skin of the user during use. For purposes herein, penetration hardness is the needle penetration in millimeters according to ASTM D 1321, Needle Penetration of Petroleum Waxes. Lower needle penetration hardness values correspond to harder materials. The penetration hardness of the formulations of this invention can be from about 5 to 360 millimeters, more specifically from about 5 to about 200 millimeters, more specifically from about 20 to about 150 millimeters, and still more specifically from about 40 to about 140 millimeters, and more specifically from about 60 to about 120 millimeters. (Formulations having a needle penetration hardness greater than 360 millimeters cannot be measured using ASTM method D 1321). The hardness of the lotion formulations described herein is important for at least two reasons. First, the softer the formulation, the more mobile the formulation will be, making the formulation more likely to migrate to the interior of the absorbent product, which, as discussed above, is not desirable. Second, softer formulations tend to be more greasy/oily to the touch, which is also less desirable. In general, formulations having a needle penetration hardness of from about 200 to 360 millimeters feel creamy to slightly greasy with less smoothness (depending on additives). Formulations that have needle penetration hardness values of from about 5 to about 200 millimeters feel silky to creamy and very smooth (depending on additives).

Along with the components described above, an optional hydrophilic surfactant may be added to the lotion formulations described herein to enhance the wettability of the treated absorbent product. Depending upon the composition of the lotion formulation, and specifically which structurant is utilized, it may be advantageous to add a hydrophilic surfactant to ensure that the absorbent product has sufficient wettability upon use.

Suitable hydrophilic surfactants should be miscible with the emollient, structurant, and rheology enhancer so as to form a substantially homogeneous mixture. Desirably, the hydrophilic surfactant should be mild and substantially non-irritating to skin such that individuals with sensitive skin can easily use the product comprising the lotion formulation. Generally, the hydrophilic surfactant will be a nonionic surfactant to be not only non-irritating to the skin, but also to avoid other undesirable affects on the absorbent product.

Suitable nonionic surfactants should be substantially nonmigratory after the lotion formulation is applied to the absorbent product. Typically, the nonionic surfactant will have a hydrophilic/lipophilic balance value in the range from about 4 to about 20, preferably from about 2 to about 7. It is also advantageous for the nonionic surfactant to have a melting point greater than about 30° C. to ensure stability in the product.

Nonionic surfactants suitable for incorporation into the lotion formulations described herein include alkylglycosides, alkylglycoside ethers, alkylpolyethoxylated esters, ethoxylated sorbitan mono-, di-, and/or trimesters of C₁₂-C₁₈ fatty acids having an average degree of ethoxylation of from about 2 to about 20, and silicone copolymers. The lotion formulation may comprise from about 0.1% (by total weight of the formulation) to about 20% (by total weight of the formulation), desirably from about 1% (by total weight of the formulation) to about 10% (by total weight of the formulation) of the hydrophilic surfactant.

In order to better enhance the benefits to consumers, additional ingredients can be incorporated into the lotion formulation described herein. The classes of ingredients and their corresponding benefits include, without limitation: antifoaming agents (reduce the tendency of foaming during processing); antimicrobial actives; antifungal actives; antiseptic actives; antioxidants (product integrity to prevent oxidation of natural oils and other ingredients on the formulation or composition); astringents--cosmetic (induce a tightening or tingling sensation on skin); astringents--drug (a drug product which checks oozing, discharge, or bleeding when applied to skin or mucous membrane and works by coagulating protein); biological additives (enhance the performance or consumer appeal of the product including vitamins); colorants (impart color to the product); antiviral actives; deodorants (reduce or eliminate unpleasant odor and protect against the formation of malodor on body surfaces); film formers (to hold active ingredients on the skin by producing a continuous film on skin upon drying); fragrances (consumer appeal and odor masking); humectants such as glycerin, lubricants, such as silicones and organomodified silicones; natural moisturizing agents (NMF) and other skin moisturizing ingredients known in the art; skin conditioning agents; skin exfoliating agents (ingredients that increase the rate of skin cell turnover such as alpha hydroxy acids and beta hydroxyacids); skin protectants (a drug product which protects injured or exposed skin or mucous membrane surface from harmful or annoying stimuli); solvents (liquids employed to dissolve components found useful in the cosmetics or drugs); and UV absorbers.

### EXAMPLE 1

In this Example, several lotion formulations were prepared and evaluated for penetration hardness, viscosity at 55° C. (1/sec) and viscosity at 60° C. (1/sec). The composition of each of the lotion formulations tested are set forth in the tables below, along with the hardness and viscosity results.

TABLE 1

| Component | A Wt. % | B Wt. % | C Wt. % | D Wt. % |
|---|---|---|---|---|
| Petrolatum | 76.00 | 78.00 | 76.00 | 83.00 |
| Alpha Olefin | 12.00 | 7.00 | 3.00 | 3.00 |
| Polymer (C₂₄- | | | | |
| C₂₈) | | | | |
| Ethylene/Vinyl | 12.00 | 15.00 | 21.00 | 14.00 |
| Acetate | | | | |
| Copolymer | | | | |
| with | | | | |
| Polyethylene | | | | |
| Hardness | 75 | 110 | 88 | 71 |
| Viscosity @ | 17,100 | 23,000 | 63,500 | 10,200 |
| 55° C., 0.5 | | | | |
| 1/sec | | | | |
| Viscosity @ | 171 | 206 | 2990 | 1670 |
| 60° C., 0.5 | | | | |
| 1/sec | | | | |

TABLE 2

| | A Wt. | B Wt. | C Wt. | D Wt. | E Wt. | F Wt. |
|---|---|---|---|---|---|---|
| Component | % | % | % | % | % | % |
| Petrolatum | 80.00 | 80.00 | 78.00 | 77.00 | 80.00 | 79.00 |
| Polyethylene | 13.00 | 15.00 | 15.00 | 18.00 | 15.00 | 15.00 |
| and | | | | | | |
| Ethylene/ | | | | | | |
| Vinyl | | | | | | |
| Acetate | | | | | | |
| Copolymer | | | | | | |
| Alpha | 7.00 | 5.00 | 7.00 | 5.00 | 3.00 | 3.00 |
| Olefin | | | | | | |
| Polymer | | | | | | |
| (C₂₄-C₂₈) | | | | | | |
| Ethylene/ | 0.00 | 0.00 | 0.00 | 0.00 | 2.00 | 3.00 |
| Vinyl | | | | | | |
| Acetate | | | | | | |
| Copolymer | | | | | | |
| (ELVAX 410 | | | | | | |
| Resin) | | | | | | |
| Hardness | 76 | 91 | 80 | 83 | 95 | 83 |
| Viscosity @ | 5310 | 13,200 | 20,630 | 171,700 | 12,010 | 236,200 |
| 55° C., 0.5 | | | | | | |
| 1/sec | | | | | | |
| Viscosity @ | 1180 | 595 | 814 | 1153 | 2663 | 1427 |
| 60° C., | | | | | | |
| 0.5 1/sec | | | | | | |

TABLE 3

| Component | A Wt. % | B Wt. % | C Wt. % | D Wt. % |
|---|---|---|---|---|
| Petrolatum | 81.00 | 75.00 | 80.00 | 80.00 |
| Ethylene/Vinyl | 0.00 | 0.00 | 2.00 | 2.00 |
| Acetate | | | | |
| Copolymer | | | | |
| (ELVAX 410 | | | | |
| Resin) | | | | |
| Polyethylene | 12.00 | 10.00 | 15.00 | 15.00 |
| and | | | | |
| Ethylene/Vinyl | | | | |
| Acetate | | | | |
| Copolymer | | | | |
| Hydrogenated | 0.00 | 15.00 | 3.00 | 0.00 |
| Cottonseed | | | | |
| Oil | | | | |
| Hardness | 88 | 102 | 86 | 86 |
| Viscosity @ | 3184 | 1591 | 18,430 | 8591 |
| 55° C., 0.5 | | | | |
| 1/sec | | | | |
| Viscosity @ | 169 | 44 | 1846 | 1105 |
| 60° C., 0.5 | | | | |
| 1/sec | | | | |

TABLE 4

| Component | A Wt. % | B Wt. % | C Wt. % |
|---|---|---|---|
| Petrolatum | 75 | 0 | 0 |
| Versagel PT200 | 0 | 80 | 75 |
| (Petrolatum and | | | |
| styrene | | | |
| copolymer) | | | |
| Ethylene/Vinyl | 10 | 0 | 10 |
| Acetate Copolymer | | | |
| and Polyethylene | | | |
| Stearyl Behenate | 15 | 20 | 15 |
| Hardness | Not Done | 76 | 92 |
| Viscosity @ 55° C., | <50 | 7622 | 3130 |
| 0.5 1/sec | | | |
| Viscosity @ 60° C., | <50 | 7078 | 2593 |
| 0.5 1/sec | | | |

In view of the above, it will be seen that the several objects of the invention are achieved. As various changes could be made in the above-described articles and products without departing from the scope of the invention, it is intended that all matter contained in the above description be interpreted as illustrative and not in a limiting sense.

1. An absorbent article comprising a liner material having a bodyfacing surface, the bodyfacing surface having deposited thereon a lotion formulation in an amount of from about 0.05 mg/cm² to about 100 mg/cm² and comprising from about 10% (by total weight of the formulation) to about 89% (by total weight of the formulation) of an emollient, from about 10% (by total weight of the formulation) to about 50% (by total weight of the formulation) of a structurant, and from about 0.1% (by total weight of the formulation) to about 40% (by total weight of the formulation) of a rheology enhancer, the rheology enhancer being selected from the group consisting of dextrin palmitate, dextrin palmitate ethylhexanoate, stearoyl inulin, combinations of alpha-olefins and styrene alone or in combination with mineral oil or petrolatum, combinations of di-functional alpha-olefins and styrene alone or in combination with mineral oil or petrolatum, combinations of alpha-olefins and isobutene alone or in combination with mineral oil or petrolatum, ethylene/propylene/styrene copolymers alone or in combination with mineral oil or petrolatum, butylene/ethylene/styrene copolymers alone or in combination with mineral oil or petrolatum, styrene/butadiene/styrene copolymers, styrene/isoprene/styrene copolymers, styrene-ethylene/butylene-styrene copolymers, styrene-ethylene/propylene-styrene copolymers, (styrene-butadiene)n polymers, (styrene-isoprene)n polymers, styrene-butadiene polymers, styrene-ethylene/propylene copolymers, polyethylene polyisobutylenes, polyisobutylenes, polyisobutenes, and combinations thereof.

2. The absorbent article as set forth in claim 1 wherein the emollient is present in an amount of from about 30% (by total weight of the formulation) to about 80% (by total weight of the formulation).

3. The absorbent article as set forth in claim 1 wherein the emollient is present in an amount of from about 60% (by total weight of the formulation) to about 80% (by total weight of the formulation).

4. The absorbent article as set forth in claim 1 wherein the structurant is present in an amount of from about 20% (by total weight of the formulation) to about 40% (by total weight of the formulation).

5. The absorbent article as set forth in claim 1 wherein the rheology enhancer is present in an amount of from about 0.5% (by total weight of the formulation) to about 30% (by total weight of the formulation).

6. The absorbent article as set forth in claim 1 wherein the rheology enhancer is present in an amount of from about 1% (by total weight of the formulation) to about 25% (by total weight of the formulation).

7. The absorbent article as set forth in claim 1 wherein the lotion formulation has a melt point viscosity of from about 5000 cPs to about 1,000,000 cPs.

8. The absorbent article as set forth in claim 1 wherein the lotion formulation has a melt point viscosity of from about 50,000 cPs to about 800,000 cPs.

9. The absorbent article as set forth in claim 1 wherein the lotion formulation has a melt point viscosity of from about 100,000 cPs to about 500,000 cPs.

10. The absorbent article as set forth in claim 1 wherein the lotion formulation has a process temperature viscosity of from about 50 cPs to about 50,000 cPs.

11. The absorbent article as set forth in claim 1 wherein the lotion formulation has a process temperature viscosity of from about 75 cPs to about 10,000 cPs.

12. The absorbent article as set forth in claim 1 wherein the lotion formulation has a process temperature viscosity of from about 100 cPs to about 5,000 cPs.

13. The absorbent article as set forth in claim 1 wherein the lotion formulation further comprises an additional ingredient selected from the group consisting of antifoaming agents, antimicrobial actives, antiviral actives, humectants, antifungal actives, antiseptic actives; antioxidants, cosmetic astringents, drug astringents, biological additives, colorants, deodorants, film formers, fragrances, lubricants, natural moisturizing agents, skin conditioning agents, skin exfoliating agents, skin protectants, solvents, hydrophilic surfactants, and UV absorbers.

14. The absorbent article as set forth in claim 1 wherein emollient is selected from the group consisting of petrolatum, mineral oil, mineral jelly, isoparaffins, vegetable oils, avocado oil, borage oil, canola oil, castor oil, chamomile, coconut oil, corn oil, cottonseed oil, evening primrose oil, safflower oil, sunflower oil, soybean oil, sweet almond, lanolin, partially hydrogenated vegetable oils, polydimethylsiloxanes, methicone, cyclomethicone, dimethicone, dimethiconol, and trimethicone, organo-siloxanes silicone elastomers, gums, resins, fatty acid esters glyceryl esters and derivatives, fatty acid ester ethoxylates, alkyl ethoxylates, C₁₂-C₂₈ fatty alcohols, C₁₂-C₂₈ fatty acids, C₁₂-C₂₈ fatty alcohol ethers, Guerbet alcohols, Guerbet Acids, Guerbet Esters, and combinations thereof.

15. The absorbent article as set forth in claim 1 wherein the structurant has a melting point of from about 45° C. to about 85° C.

16. The absorbent article as set forth in claim 1 wherein the structurant is selected from the group consisting of animal waxes, vegetable waxes, mineral waxes, synthetic waxes, polymers, bayberry wax, beeswax C₃₀ alkyl dimethicone, candelilla wax, carnauba, ceresin, cetyl esters, stearyl benzoate, behenyl benzoate, esparto, hydrogenated cottonseed oil, hydrogenated jojoba oil, hydrogenated jojoba wax, hydrogenated microcrystalline wax, hydrogenated rice bran wax, japan wax, jojoba buffer, jojoba esters, jojoba wax, lanolin wax, microcrystalline wax, mink wax, motan acid wax, motan wax, ouricury wax, ozokerite paraffin, PEG-6 beeswax, PEG-8 beeswax, rezowax, rice bran wax, shellac wax, spent grain wax, spermaceti wax, steryl dimethicone, synthetic beeswax, synthetic candelilla wax, synthetic carnauba wax, synthetic japan wax, synthetic jojoba wax, C₁₄-C₂₈ fatty alcohols, C₁₄-C₂₈ fatty acids, polyethylene, ethylene vinyl acetate copolymers,ethylene-alpha olefin copolymers, ethylene homopolymers, C₁₈-C₄₅ olefins, poly alpha olefins, hydrogenated vegetable oils, polyhydroxy fatty acid esters, polyhydroxy fatty acid amides, ethoxylated fatty alcohols and esters of C₁₂-C₂₈ fatty acids, C₁₂-C₂₈ fatty alcohols and combinations thereof.

17. The absorbent article as set forth in claim 1 wherein the lotion formulation is present in an amount of from about 1 mg/cm² to about 50 mg/cm².

18. The absorbent article as set forth in claim 1 wherein the lotion formulation is present in an amount of from about 10 mg/cm² to about 40 mg/cm².

19. The absorbent article as set forth in claim 1 wherein the lotion formulation has a penetration hardness of from about 40 to about 140.

20. The absorbent article as set forth in claim 1 wherein the lotion formulation has a penetration hardness of from about 60 to about 120.

21. An absorbent article comprising a liner material having a bodyfacing surface, the bodyfacing surface having deposited thereon a lotion formulation in an amount of from about 0.05 mg/cm² to about 100 mg/cm² and comprising from about 10% (by total weight of the formulation) to about 89% (by total weight of the formulation) of an emollient, from about 10% (by total weight of the formulation) to about 50% (by total weight of the formulation) of a structurant, and from about 1% (by total weight of the formulation) to about 40% (by total weight of the formulation) of a rheology enhancer, wherein the lotion formulation has a melt point viscosity of from about 5000 cPs to about 1,000,000 cPs and a process temperature viscosity of from about 50 cPs to about 50,000 cPs, the rheology enhancer being selected from the group consisting of dextrin palmitate, dextrin palmitate ethylhexanoate, stearoyl inulin, combinations of alpha-olefins and styrene alone or in combination with mineral oil or petrolatum, combinations of di-functional alpha-olefins and styrene alone or in combination with mineral oil and petrolatum, combinations of alpha-olefins and isobutene alone or in combination with mineral oil or petrolatum, ethylene/propylene/styrene copolymers alone or in combination with mineral oil or petrolatum, butylene/ethylene/styrene copolymers alone or in combination with mineral oil or petrolatum, styrene/butadiene/styrene copolymers, styrene/isoprene/styrene copolymers, styrene-ethylene/butylene-styrene copolymers, styrene-ethylene/propylene-styrene copolymers, (styrene-butadiene)n polymers, (styrene-isoprene)n polymers, styrene-butadiene polymers, styrene-ethylene/propylene copolymers, polyethylene polyisobutylenes, polyisobutylenes, polyisobutenes, and combinations thereof.

22. The absorbent article as set forth in claim 21 wherein the emollient is present in an amount of from about 30% (by total weight of the formulation) to about 80% (by total weight of the formulation).

23. The absorbent article as set forth in claim 21 wherein the emollient is present in an amount of from about 60% (by total weight of the formulation) to about 80% (by total weight of the formulation).

24. The absorbent article as set forth in claim 21 wherein the structurant is present in an amount of from about 20% (by total weight of the formulation) to about 40% (by total weight of the formulation).

25. The absorbent article as set forth in claim 21 wherein the rheology enhancer is present in an amount of from about 0.5% (by total weight of the formulation) to about 30% (by total weight of the formulation).

26. The absorbent article as set forth in claim 21 wherein the rheology enhancer is present in an amount of from about 1% (by total weight of the formulation) to about 25% (by total weight of the formulation).

27. The absorbent article as set forth in claim 21 wherein the melt point viscosity is from about 50,000 cPs to about 800,000 cPs.

28. The absorbent article as set forth in claim 21 wherein the melt point viscosity is from about 100,000 cPs to about 500,000 cPs.

29. The absorbent article as set forth in claim 21 wherein the process temperature viscosity is from about 75 cPs to about 10,000 cPs.

30. The absorbent article as set forth in claim 21 wherein the process temperature viscosity is from about 100 cPs to about 5,000 cPs.

31. The absorbent article as set forth in claim 21 wherein emollient is selected from the group consisting of petrolatum, mineral oil, mineral jelly, isoparaffins, vegetable oils, avocado oil, borage oil, canola oil, castor oil, chamomile, coconut oil, corn oil, cottonseed oil, evening primrose oil, safflower oil, sunflower oil, soybean oil, sweet almond, lanolin, partially hydrogenated vegetable oils, polydimethylsiloxanes, methicone, cyclomethicone, dimethicone, dimethiconol, and trimethicone, organo-siloxanes silicone elastomers, gums, resins, fatty acid esters glyceryl esters and derivatives, fatty acid ester ethoxylates, alkyl ethoxylates, C₁₂-C₂₈ fatty alcohols, C₁₂-C₂₈ fatty acids, C₁₂-C₂₈ fatty alcohol ethers, Guerbet alcohols, Guerbet Acids, Guerbet Esters, and combinations thereof.

32. The absorbent article as set forth in claim 21 wherein the structurant has a melting point of from about 45° C. to about 85° C.

33. The absorbent article as set forth in claim 21 wherein the structurant is selected from the group consisting of animal waxes, vegetable waxes, mineral waxes, synthetic waxes, polymers, bayberry wax, beeswax C₃₀ alkyl dimethicone, candelilla wax, carnauba, ceresin, cetyl esters, stearyl benzoate, behenyl benzoate, esparto, hydrogenated cottonseed oil, hydrogenated jojoba oil, hydrogenated jojoba wax, hydrogenated microcrystalline wax, hydrogenated rice bran wax, japan wax, jojoba buffer, jojoba esters, jojoba wax, lanolin wax, microcrystalline wax, mink wax, motan acid wax, motan wax, ouricury wax, ozokerite paraffin, PEG-6 beeswax, PEG-8 beeswax, rezowax, rice bran wax, shellac wax, spent grain wax, spermaceti wax, steryl dimethicone, synthetic beeswax, synthetic candelilla wax, synthetic carnauba wax, synthetic japan wax, synthetic jojoba wax, C₁₄-C₂₈ fatty alcohols, C₁₄-C₂₈ fatty acids, polyethylene, ethylene vinyl acetate copolymers, ethylene-alpha olefin copolymers, ethylene homopolymers, C₁₈-C₄₅ olefins, poly alpha olefins, hydrogenated vegetable oils, polyhydroxy fatty acid esters, polyhydroxy fatty acid amides, ethoxylated fatty alcohols and esters of C₁₂-C₂₈ fatty acids, C₁₂-C₂₈ fatty alcohols and combinations thereof.

34. The absorbent article as set forth in claim 21 wherein the lotion formulation further comprises an additional ingredient selected from the group consisting of antifoaming agents, antimicrobial actives, antiviral actives, humectants, antifungal actives, antiseptic actives; antioxidants, cosmetic astringents, drug astringents, biological additives, colorants, deodorants, film formers, fragrances, lubricants, natural moisturizing agents, skin conditioning agents, skin exfoliating agents, skin protectants, solvents, hydrophilic surfactants, and UV absorbers.

35. The absorbent article as set forth in claim 21 wherein the lotion formulation has a penetration hardness of from about 40 to about 140.

36. The absorbent article as set forth in claim 21 wherein the lotion formulation has a penetration hardness of from about 60 to about 120.

37. An absorbent article comprising a liner material having a bodyfacing surface, the bodyfacing surface having deposited thereon a lotion formulation in an amount of from about 0.05 mg/cm² to about 100 mg/cm² and comprising from about 10% (by total weight of the formulation) to about 89% (by total weight of the formulation) of an emollient, from about 10% (by total weight of the formulation) to about 50% (by total weight of the formulation) of a structurant, and from about 0.1% (by total weight of the formulation) to about 40% (by total weight of the formulation) of Versagel.
